(19) United States
(12) Patent Application Publication
(10) Pub. No.: US 2007/0016161 A1
(43) Pub. Date: Jan. 18, 2007

(54) Controlled flow adapter with piercing end for medical fluid containers

(75) Inventors: Mark Paul Costa, Mississauga (CA); Philip J. Simpson, Escondido, CA (US); David G. Matsuura, Encinitas, CA (US)

Correspondence Address: BUCHANAN, INGERSOLL & ROONEY PC, POST OFFICE BOX 1404, ALEXANDRIA, VA, 22313-1404, US

(73) Assignee: Vasogen Ireland Limited

(21) Appl. No.: 11/450,268

(22) Filed: Jun. 12, 2006

Related U.S. Application Data
(60) Provisional application No. 60/690,462, filed on Jun. 13, 2005.

(51) Int. Cl. A61M 5/32

(52) U.S. Cl. 604/411

(57) ABSTRACT

A non-luer apparatus for controlled flow of medical fluid materials for use as an adapter in a covered outlet of a medical fluid container, has a body with a hollow interior, a first open end adapted for attachment with the medical fluid container outlet, and a second open end adapted for fit with a medical accessory. The first open end also has a piercing portion for piercing the covered outlet. The second open end further includes an upstanding circumferential wall and associated retaining means for releasable attachment with the medical accessory. A fluid flow control means is disposed within the body to control the flow of medical fluid materials between the medical fluid container and the medical accessory where the fluid flow control means is motivated to open or close fluid communication when the medical accessory is attached or disengaged from the second open end.

This application claims priority under 35 U.S.C. § 119(e) to U.S. Provisional Application No. 60/690,462 filed on Jun. 13, 2005.

### FIELD OF THE INVENTION

The present invention relates to an apparatus for use with medical fluid containers for transfer of medical fluid materials, and more particularly a non-luer controlled flow adapter having a piercing end to pierce the sealed outlet of a medical fluid container.

### BACKGROUND ART

Many different types of fluids are used by doctors and medical professionals for treating patients, surgeries, and other therapies. For example, drugs can be stored in bottles or vials, and fluids such as physiological saline are typically stored into bags or bottles. Accordingly, these fluids are placed in containers that maintain sterility but also permit access to the fluids by the medical professional or user. Typically, the outlet end of these containers (i.e. bottle, vial, or bag) comprises an elastomeric septum or piercable cover that is pierced by a needle or similar device attached to a medical accessory for insertion/withdrawal of the medical fluid to/from the container. For example, infusion fluids such as physiological saline or Ringer's lactate, are stored in sealed bags made of polyvinyl chloride and often the bags have two ports. One port is for inserting drugs or other materials into the bag and consequent mixing with the infusion fluid, such that the patient will be infused with the drug or other material. The other port is for accessing the infusion fluid. Typically, these ports will be covered with either a septum or other material to maintain sterility of the fluid contents. Adapters with piercing elements are often used to access the fluid contents, whereby the adapters also have outlets that can attach to medical accessories to permit fluid flow from the medical fluid container to the medical accessory.

Often there is the need for multiple accesses to the medical fluid from the container (i.e. infusion fluids, drug vials, etc.) and the adapter is required to permit such multiple accesses. One method for permitting multiple accesses is to close off the outlet end of the container using a clamp to prevent fluid flow through the adapter. Another way of permitting multiple accesses is to have a self-sealing valve incorporated in the adapter that permits fluid communication with the medical fluid container whereby the valve is actuated when a medical accessory is attached to the adapter and subsequently self-seals when the medical accessory is removed. Typically, the adapter has a luer fitting to allow attachment of the adapter to a complementary luer fitting on a medical accessory.

Accordingly, it is an object of the present invention to provide a novel controlled flow adapter having a piercing end to pierce the sealed outlets of medical fluid containers, where there is a non-luer fitting for attachment with a medical accessory.

### SUMMARY OF THE INVENTION

According to one embodiment of this invention, a controlled flow adapter with a piercing end for piercing the covered outlet of a medical fluid container is provided, comprising a body having a hollow interior and first and second open ends. The first open end is adapted to pierce and fit within the outlet of the medical fluid container. The second open end is adapted for fit with a medical accessory for flow of medical fluid materials from the medical fluid container to the medical accessory and is not shaped as a luer fitting. A fluid flow control means is disposed within the body for controlling the flow of medical fluid materials between the hollow interior of the medical fluid container and the medical accessory by way of the controlled flow adapter. The fluid flow control means comprises a valve member, a valve seat, and biasing means operating on the valve member to bias the valve member against the valve seat for fluid tight fit. The valve member is motivated to open fluid communication between the medical fluid container and the medical accessory by attachment of the medical accessory to the second end of the body and the valve member is motivated to close fluid communication between the medical fluid container and the medical accessory when the medical accessory is disengaged from the second end of the body.

Suitably, the biasing means comprises a spring. The second end for fitting with the medical accessory includes an upstanding circumferential wall, and a retaining means for releasably attaching the medical accessory to the second end. Suitably, the attachment means comprises at least one bayonet slot.

The valve member may include a valve nose extending axially outwardly from the valve seat for engagement with the medical accessory to open fluid communication. Suitably, the valve seat includes an upstanding annular ring on the surface distal to the outlet of the medical fluid container for fluid tight sealing engagement with the medical accessory.

According to another embodiment of the invention, another valve is placed in series with the fluid control means to govern unidirectional flow of fluid to or from the medical fluid container. This valve can be a check valve.

In yet another embodiment, a gas vent port is placed in parallel with the hollow interior of the controlled flow adapter to allow for exchange of gases between the medical fluid container and the environment without compromising sterility or allowing fluid to escape to the environment. Suitable membranes can be utilized in association with the gas vent port to prevent fluid escape and to maintain sterility of the medical fluid. Such membranes can include hydrophobic membranes and membranes which prevent passage of viruses and bacteria.

A specific preferred embodiment of the invention is illustrated in the accompanying drawings.

### BRIEF DESCRIPTION OF THE DRAWINGS

FIG. 1 is a perspective view of the preferred embodiment of the invention;

FIG. 2 is a side view of the apparatus of FIG. 1; and

FIG. 3 is a side cross-sectional view of the apparatus of FIG. 1 engaged at one end with a medical accessory.

### DESCRIPTION OF THE PREFERRED EMBODIMENT

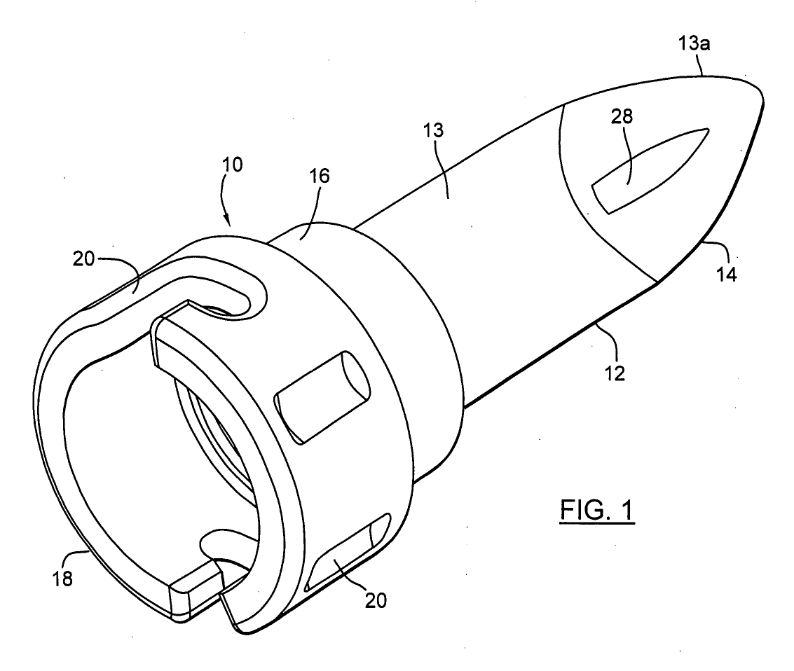

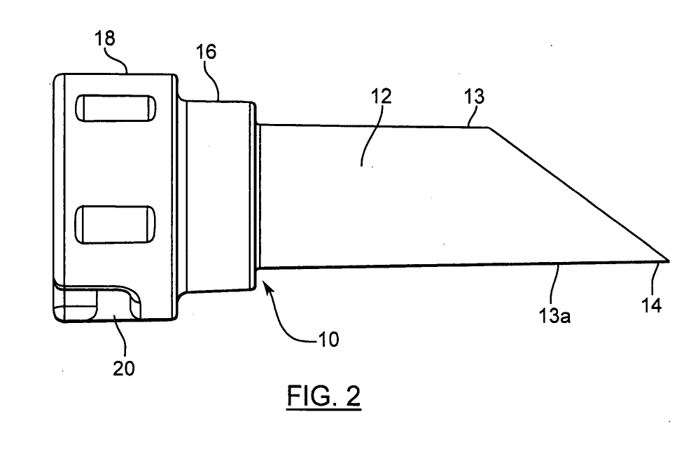

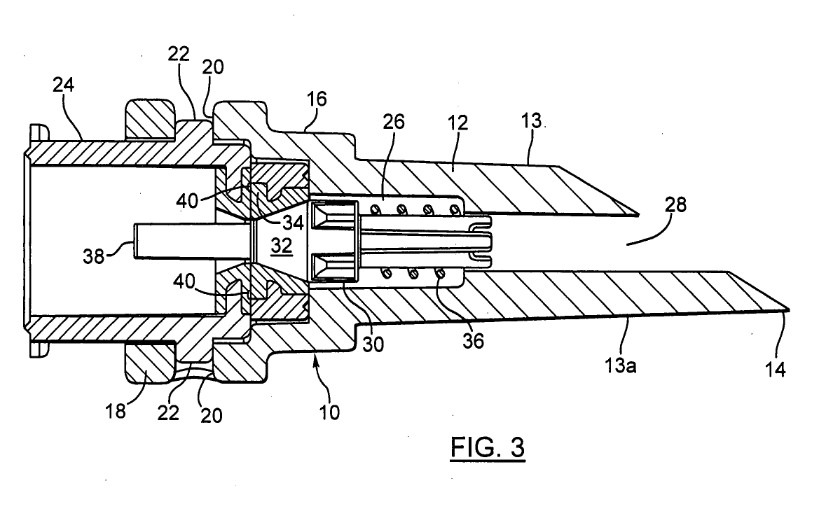

FIGS. 1 to 3 show a controlled flow adapter having a piercing end according to the preferred embodiment of the invention. As shown in FIGS. 1 and 2, the controlled flow apparatus 10 has an inlet housing 12 adapted to pierce through and fit within a covered outlet of a medical fluid container (not shown). Inlet housing 12 has a piercing end 14 that pierces through the covered outlet of a medical fluid container. The upper side 13 of inlet housing 12 is shorter than the bottom side 13a of inlet housing 12. A circumferential wall 16 depends from inlet housing 12 and has an outer diameter greater than the outer diameter of inlet housing 12. Circumferential wall 16 abuts against the end of the outlet of the medical fluid container distal to the container portion of the medical fluid container (not shown) when controlled flow adapter 10 is inserted into the outlet. An outlet housing 18 depends from circumferential wall 16 and has an outer diameter greater than the outer diameter of circumferential wall 16. Outlet housing 18 has bayonet slots 20 for fit with corresponding bayonet pins 22 on a medical accessory 24 (see FIG. 3) to releasably attach the medical accessory 24 to the controlled flow adapter 10.

As seen in FIG. 3, the controlled flow adapter 10 has a hollow interior 26 through which medical fluid materials will flow from a medical fluid container (not shown) by way of an axial bore 28 within inlet housing 12. A valve 30 including a valve head 32 and a valve seat 34 is disposed within hollow interior 26, in association with a spring 36. Spring 36 biases valve head 32 against valve seat 34. Valve head 32 also has an axially extending valve nose 38 which, in operation, will engage with a valve actuating member (not shown) associated with medical accessory 24 with subsequent movement of valve 30 in a rearward direction towards the medical fluid container, thereby unseating valve head 32 from valve seat 34 to provide for fluid communication from the medical container to medical accessory 24. When medical accessory 24 is disengaged from outlet housing 18, spring 36 biases valve head 32 against valve seat 34 to provide for a fluid tight seal to prevent medical fluid materials from exiting controlled flow adapter 10 when medical accessory 24 is disengaged.

Valve seat 34 is preferably made of elastomeric material but can be made of any suitable material known in the art. An upstanding annular ring 40 is disposed on the surface of valve seat 34 distal to the outlet of the medical fluid container and assists in creating a fluid tight seal between valve seat 34 and medical accessory 24 when medical accessory 24 is attached to outlet housing 18, thereby reducing the risk that medical fluid will leak from the controlled flow adapter 10—medical accessory 24 interface area.

As can be appreciated, there can be modifications to the structures and functions described above. Although bayonet slots 22 are described for releasably attaching medical accessory 24 with outlet housing 18, any suitable means for releasably attaching medical accessory 24 with outlet housing 18 can be used. Interference fits, snap fits, taper fits, threads, or magnets can be used as means for releasably attaching medical accessory 24 to outlet housing 18. Further, medical fluid containers can encompass any container suitable for holding or storing medical fluids such as bottle, vials, or bags made of elastomeric materials, and can encompass containers which contain lyophilized or powdered drugs which can be reconstituted with the addition of water or any suitable liquid. Medical fluid materials can encompass any fluids used for medical purposes or in a medical setting, such as physiological saline, liquid pharmaceutical products, and liquid nutritional supplements, and includes lyophilized or powdered materials that are reconstituted by the addition of water or any suitable liquid. Medical accessories can encompass any suitable device for the transfer and/or storage of medical fluid materials to or from a medical fluid container such as syringes, pumps, bottles, vials or the like.

Although a spring 36 has been described as the means to bias valve head 32 against valve seat 34, any suitable biasing means can be used including gas springs, cantilever beams, and elastomeric materials. The valve member may also comprise an unitary elastomeric member that seals the hollow interior of the body which deflects or otherwise deforms to permit fluid communication between the medical fluid container and the medical accessory when the medical accessory is attached to the adapter.

The end of the controlled flow adapter that is adapted to pierce the end of the medical fluid container can be dimensioned as any suitable piercing configuration, including symmetrical or asymmetrical cylindrical tapered points or a prismatic piercing point (multiple bevels that form a point).

In addition, inlet housing 12 can also have attachment means to prevent disengagement of the controlled flow adapter from the medical fluid container. The attachment means can include at -least one radially outwardly extending protuberance/flange/arm which engage the inner surface of the outlet of the medical fluid container, and at least one rearwardly angled arm or flange which resists movement of the controlled flow adapter in a rearward direction after insertion of the inlet housing 12 into the outlet of the medical fluid container. In addition, the adapter can have an associated engagement means which can comprise at least one arm having gripping means to grip the outer surface of the outlet to prevent disengagement of the adapter from the medical fluid container. The gripping means can comprise a tooth that grips the outer surface of the outlet or engages a slot in the outer surface of the outlet.

What is claimed is:

1. A non-luer apparatus for controlled flow of medical fluid materials for use as an adapter in an outlet of a medical fluid container wherein said outlet has a piercable cover, comprising:
a body having a hollow interior, a first open end adapted for attachment with said outlet of said medical fluid container, and a second open end adapted for fit with a medical accessory;
said first open end having a piercing portion to pierce through said cover of said medical fluid container;
said second end further including an upstanding circumferential wall and retaining means associated with said circumferential wall for releasably attaching said medical accessory to said second end;
a fluid flow control means disposed within said body for controlling the flow of medical fluid materials through said hollow interior between said medical fluid container and said medical accessory; and
said fluid flow control means comprising a valve member, a valve seat, biasing means operating on the valve member to bias the valve member against the valve seat for fluid tight fit, and whereby the valve member is motivated to open fluid communication between said medical fluid container and said medical accessory by attachment of said medical accessory to said second open end and said valve member is motivated to close fluid communication between said medical fluid container and said medical accessory when said medical accessory is disengaged from said second open end.

2. The apparatus according to claim 1, wherein the retaining means comprises at least one bayonet slot.

3. The apparatus according to claim 2, wherein said biasing means comprises a spring.

4. The apparatus according to claim 1, wherein said biasing means comprises a spring.

5. The apparatus according to claim 1, wherein said valve member further includes a valve nose extending axially outwardly from said valve seat for engagement with said medical accessory to open fluid communication.

6. The apparatus according to claim 2, wherein said valve member further includes a valve nose extending axially outwardly from said valve seat for engagement with said medical accessory to open fluid communication.

7. The apparatus according to claim 3, wherein said valve member further includes a valve nose extending axially outwardly from said valve seat for engagement with said medical accessory to open fluid communication.

8. The apparatus according to claim 1, wherein said valve seat further includes an upstanding annular ring on the surface distal to said hollow interior for fluid tight sealing engagement with said medical accessory.

9. The apparatus according to claim 2, wherein said valve seat further includes an upstanding annular ring on the surface distal to said hollow interior for fluid tight sealing engagement with said medical accessory.

10. The apparatus according to claim 3, wherein said valve seat further includes an upstanding annular ring on the surface distal to said hollow interior for fluid tight sealing engagement with said medical accessory.
